(19) United States
(12) Patent Application Publication
(10) Pub. No.: US 2024/0374188 A1
(43) Pub. Date: Nov. 14, 2024

(54) TRANSPORTATION VEHICLE, PHYSIOLOGICAL STATE DETECTION DEVICE, AND PHYSIOLOGICAL STATE DETECTION METHOD APPLIED TO TRANSPORTATION VEHICLE

(71) Applicant: ASTI GLOBAL INC., TAIWAN, Taichung City (TW)

(72) Inventor: CHIEN-SHOU LIAO, Taichung City (TW)

(21) Appl. No.: 18/657,761

(22) Filed: May 7, 2024

Related U.S. Application Data
(60) Provisional application No. 63/465,252, filed on May 10, 2023.

(51) Int. Cl. A61B 5/18; A61B 5/00; A61B 5/0205; A61B 5/1171; A61B 5/1172; A61B 5/16; B60H 3/00

(52) U.S. Cl. CPC A61B 5/18; A61B 5/0013; A61B 5/0077; A61B 5/0205; A61B 5/1172; A61B 5/1176; A61B 5/163; A61B 5/165; A61B 5/6893; A61B 5/7415; A61B 5/742; A61B 5/7455; B60H 3/0007; A61B 2560/0242; A61B 2560/0443

(57) ABSTRACT

A transportation vehicle, a physiological state detection device and a physiological state detection method are provided. The transportation vehicle configured for using the physiological state detection device includes a device casing module, a signal control module, an image capturing module and an information providing module. The image capturing module and the information providing module are disposed inside the device casing module and electrically connected to the signal control module. When the image capturing module is optionally configured to be used, the image capturing module can be allowed to be configured through the signal control module to continuously or discontinuously capture a plurality of facial images of a driver. When the information providing module is optionally configured to be used, the information providing module can be allowed to be configured through the signal control module to present a physiological state signal corresponding to the facial images.

### CROSS-REFERENCE TO RELATED PATENT APPLICATION

This application claims the benefit of priority to the U.S. Provisional Patent Application Ser. No. 63/465,252, filed on May 10, 2023, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a physiological state detection device and a physiological state detection method, and more particularly to a transportation vehicle configured for using the physiological state detection device, a physiological state detection device applied to the transportation vehicle, and a physiological state detection method applied to the transportation vehicle.

### BACKGROUND OF THE DISCLOSURE

In the related art, a driver can monitor the driver's own health states and perform health management through physiological data and long-term records obtained by a physiological detection device. However, the physiological detection device in the related art still has room for improvement.

### SUMMARY OF THE DISCLOSURE

In response to the above-referenced technical inadequacy, the present disclosure provides a physiological state detection device, a transportation vehicle configured for using the physiological state detection device, and a physiological state detection method applied to the transportation vehicle.

In order to solve the above-mentioned problems, one of the technical aspects adopted by the present disclosure is to provide a physiological state detection device applied to a transportation vehicle, which includes a device casing module, a signal control module, an image capturing module, a wireless transmission module, an information providing module and a power supply module. The device casing module is configured to be disposed on a steering wheel of the transportation vehicle. The signal control module is disposed inside the device casing module. The image capturing module is disposed inside the device casing module and electrically connected to the signal control module. The wireless transmission module is disposed inside the device casing module and electrically connected to the signal control module. The information providing module is disposed inside the device casing module and electrically connected to the signal control module. The power supply module is disposed inside the device casing module and electrically connected to the signal control module. When the image capturing module is optionally configured to be used, the image capturing module is allowed to be configured through the signal control module to continuously or discontinuously capture a plurality of facial images of a driver driving the transportation vehicle within a predetermined period, thereby obtaining a plurality of facial image signals respectively corresponding to the facial images of the driver. When the wireless transmission module is optionally configured to be used, the wireless transmission module is allowed to be configured through the signal control module to transmit the facial image signals to an information processing system, thereby obtaining a physiological state signal corresponding to the facial image signals. When the wireless transmission module is optionally configured to be used, the wireless transmission module is allowed to be configured through the signal control module to receive the physiological state signal that is obtained through processing by the information processing system. When the information providing module is optionally configured to be used, the information providing module is allowed to be configured through the signal control module to present the physiological state signal for reference by relevant personnel. When the power supply module is optionally configured to be used, the power supply module is allowed to be configured through the signal control module to supply power to the signal control module, the image capturing module, the wireless transmission module and the information providing module. Each of the facial images of the driver includes at least one scleral image with microvascular characteristics or at least one eyelid image with microvascular characteristics.

In order to solve the above-mentioned problems, another one of the technical aspects adopted by the present disclosure is to provide a physiological state detection device, which includes a device casing module, a signal control module, an image capturing module and an information providing module. The signal control module is disposed inside the device casing module. The image capturing module is disposed inside the device casing module and electrically connected to the signal control module. The information providing module is disposed inside the device casing module and electrically connected to the signal control module. When the image capturing module is optionally configured to be used, the image capturing module is allowed to be configured through the signal control module to continuously or discontinuously capture a plurality of facial images of a driver. When the information providing module is optionally configured to be used, the information providing module is allowed to be configured through the signal control module to present a physiological state signal corresponding to the facial images. Each of the facial images of the driver includes at least one scleral image with microvascular characteristics or at least one eyelid image with microvascular characteristics.

In order to solve the above-mentioned problems, yet another one of the technical aspects adopted by the present disclosure is to provide a transportation vehicle configured for using a physiological state detection device.

In order to solve the above-mentioned problems, yet another one of the technical aspects adopted by the present disclosure is to provide a physiological state detection method, which includes configuring a physiological state detection device on a steering wheel of a transportation vehicle; identifying an identity of a driver driving the transportation vehicle through a biometric module of the physiological state detection device; continuously or discontinuously capturing a plurality of facial images of the driver within a predetermined period by an image capturing module of the physiological state detection device, thereby obtaining blood flow changes or spectral changes in capillaries of the driver's scleras or eyelids; processing the facial images by an information processing system, thereby obtaining a physiological state signal corresponding to the facial images; and presenting the physiological state signal by an information providing module of the physiological state detection device for reference by relevant personnel. When the information providing module is allowed to be configured as an information display for displaying the physiological state signal, the information display is allowed to be configured to visually present the physiological state signal. When the information providing module is allowed to be configured as an information projector for projecting the physiological state signal to a predetermined area, the information projector is allowed to be configured to visually present the physiological state signal.

When the information providing module is allowed to be configured as a sound player for playing the physiological state signal, the sound player is allowed to be configured to audibly present the physiological state signal. When the information providing module is allowed to be configured as a vibration generator for generating different vibration frequencies based on changes in the physiological state signal, the vibration generator is configured to tangibly present the physiological state signal.

Therefore, in the physiological state detection device and the transportation vehicle provided by the present disclosure, by virtue of “the image capturing module being disposed inside the device casing module and electrically connected to the signal control module” and “the information providing module being disposed inside the device casing module and electrically connected to the signal control module,” when the image capturing module is optionally configured to be used, the image capturing module can be allowed to be configured through the signal control module to continuously or discontinuously capture a plurality of facial images (each facial image includes at least one scleral image with microvascular characteristics or at least one eyelid image with microvascular characteristics) of a driver, and when the information providing module is optionally configured to be used, the information providing module can be allowed to be configured through the signal control module to present a physiological state signal corresponding to the facial images for reference by relevant personnel.

Furthermore, in the physiological state detection method provided by the present disclosure, by virtue of “configuring a physiological state detection device on a steering wheel of a transportation vehicle,” “identifying an identity of a driver driving the transportation vehicle through a biometric module of the physiological state detection device,” “continuously or discontinuously capturing a plurality of facial images of the driver within a predetermined period by an image capturing module of the physiological state detection device, thereby obtaining blood flow changes or spectral changes in capillaries of the driver's scleras or eyelids” and “processing the facial images by an information processing system, thereby obtaining a physiological state signal corresponding to the facial images,” the physiological state signal can be presented by an information providing module of the physiological state detection device for reference by relevant personnel. For example, when the information providing module is allowed to be configured as an information display for displaying the physiological state signal, the information display can be allowed to be configured to visually present the physiological state signal for reference by relevant personnel. When the information providing module is allowed to be configured as an information projector for projecting the physiological state signal to a predetermined area, the information projector can be allowed to be configured to visually present the physiological state signal for reference by relevant personnel. When the information providing module is allowed to be configured as a sound player for playing the physiological state signal, the sound player can be allowed to be configured to audibly present the physiological state signal for reference by relevant personnel. When the information providing module is allowed to be configured as a vibration generator for generating different vibration frequencies based on changes in the physiological state signal, the vibration generator can be configured to tangibly present the physiological state signal for reference by relevant personnel.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a physiological state detection device provided by a first embodiment of the present disclosure;

FIG. 2 is a first schematic view of a transportation vehicle using the physiological state detection device provided by the present disclosure;

FIG. 3 is a schematic view of the physiological state detection device provided by the present disclosure disposed on a peripheral portion of a steering wheel of the transportation vehicle;

FIG. 4 is a second schematic view of the transportation vehicle using the physiological state detection device provided by the present disclosure;

FIG. 5 is a schematic view of the physiological state detection device provided by the present disclosure disposed on a central portion of the steering wheel of the transportation vehicle;

FIG. 6 is a schematic view of the driver's facial image captured through an image capturing module provided by the transportation vehicle using the physiological state detection device provided by the present disclosure;

FIG. 7 is a schematic enlarged view of part VII of FIG. 6;

FIG. 8 is a flowchart of the physiological state detection method provided by the first embodiment of the present disclosure; and

FIG. 9 is a functional block diagram of the physiological state detection device provided by a second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

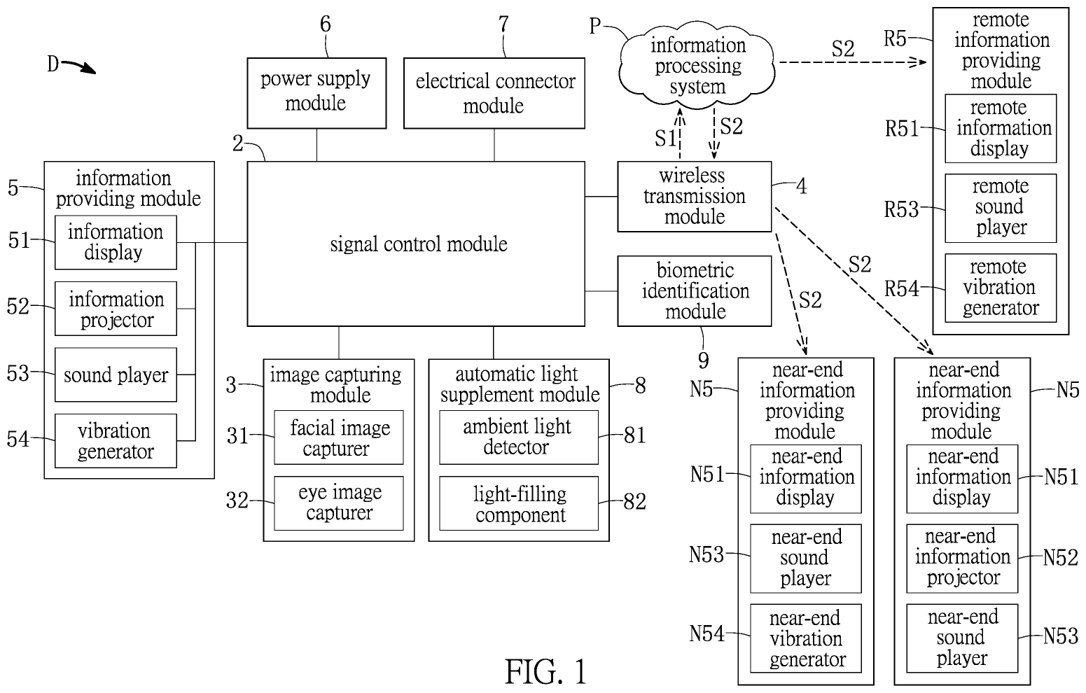

Referring to FIG. 1 to FIG. 9, the present disclosure provides a physiological state detection device D applied to a transportation vehicle T, which includes a device casing module 1, a signal control module 2, an image capturing module 3 and an information providing module 5 (that is to say, a wireless transmission module 4, a power supply module 6, an electrical connector module 7, an automatic light supplement module 8 and a biometric module 9 (or a biometric recognition module) can be omitted in the physiological state detection device D according to different requirements).

More particularly, the signal control module 2 can be disposed inside the device casing module 1, the image capturing module 3 can be disposed inside the device casing module 1 and electrically connected to the signal control module 2, and the information providing module 5 can be disposed inside the device casing module 1 and electrically connected to the signal control module 2.

Therefore, when the image capturing module 3 is optionally configured to be used, the image capturing module 3 can be allowed to be configured through the signal control module 2 to continuously or discontinuously capture a plurality of facial images M of a driver U driving the transportation vehicle T within a predetermined period. In addition, when the information providing module 5 is optionally configured to be used, the information providing module 5 can be allowed to be configured through the signal control module 2 to present a physiological state signal S2 corresponding to the facial images M for reference by relevant personnel. It should be noted that each of the facial images M of the driver U includes at least one scleral image M1 with microvascular characteristics or at least one eyelid image M2 with microvascular characteristics, so that blood flow changes (such as changes in blood flow velocity or blood flow conditions of the microvessels between the scleral images M1 or between the eyelid images M2) or spectral changes (such as absorption spectrum changes, emission spectrum changes, scattering spectrum changes or any kind of spectral changes) in the capillaries of the driver U in the scleras or eyelids can be obtained by the image capturing module 3 of the physiological state detection device D provided by the present disclosure.

### First Embodiment

Referring to FIG. 1 to FIG. 7, a first embodiment of the present disclosure provides a physiological state detection device D (or a physiological and mental state detection device, or a physiological information detection device, or a physiological and mental information detection device) that can be applied to a transportation vehicle T, which includes a device casing module 1, a signal control module 2, an image capturing module 3, a wireless transmission module 4, an information providing module 5 and a power supply module 6.

For example, the physiological state detection device D can be a portable physiological state detection device or a fixed physiological state detection device. In addition, the transportation vehicle T can be any kind of land vehicle, water vehicle, air vehicle, orbital vehicle or space vehicle that requires an operator or a driver. However, the aforementioned details are disclosed for exemplary purposes only, and are not meant to limit the scope of the present disclosure.

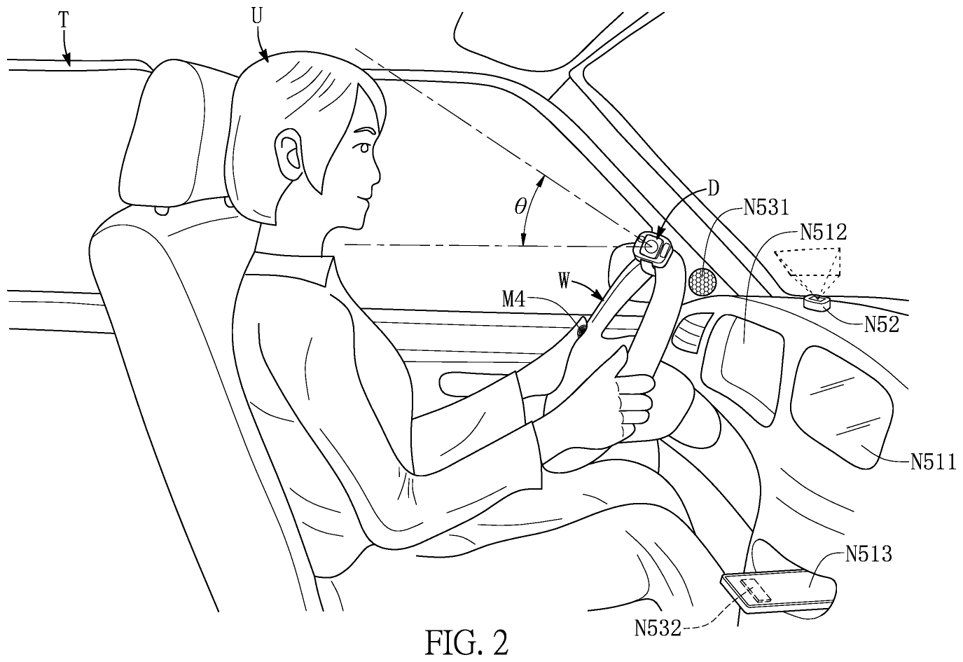

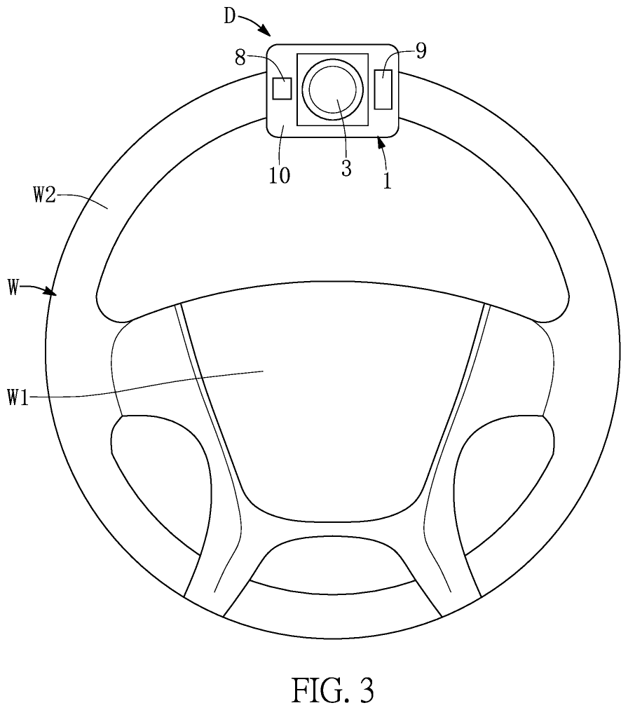

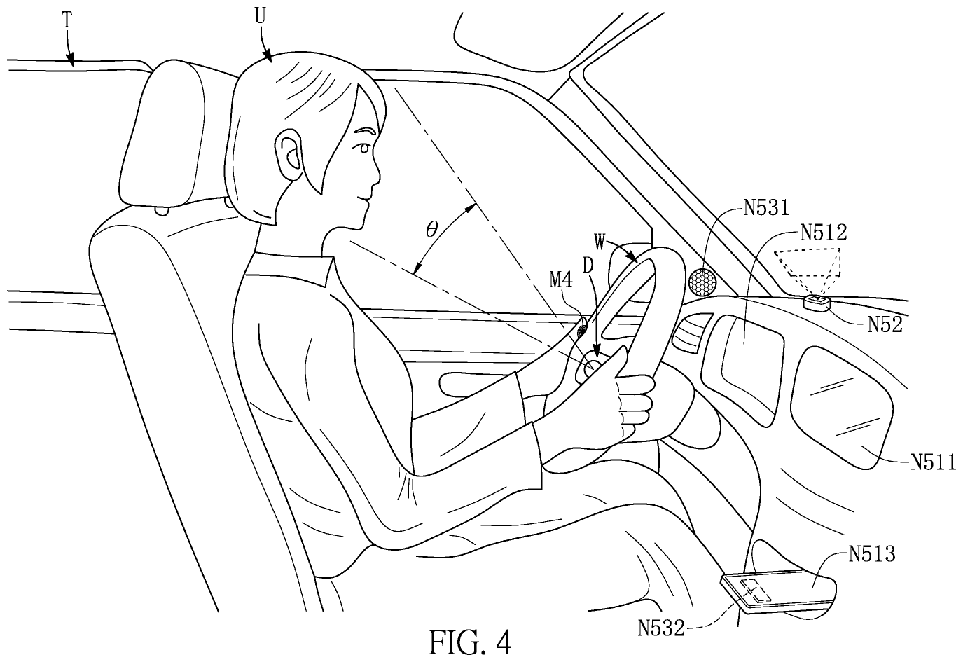

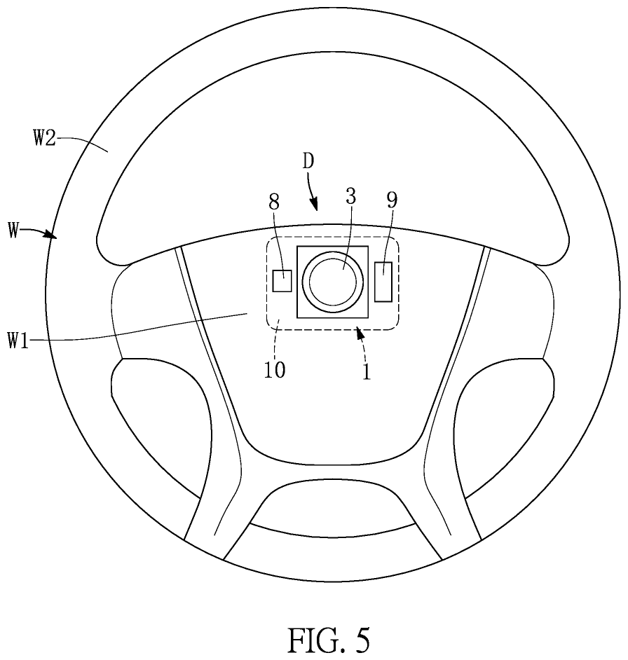

More particularly, referring to FIG. 2, FIG. 3, FIG. 4 and FIG. 5, the device casing module 1 can be configured to be disposed (or mounted) at any location on the transportation vehicle T. For example, a steering wheel W of the transportation vehicle T has a central portion W1 (or a car horn setting area) and a peripheral portion W2 (or a driver's grip area) surrounding the central portion W1. Moreover, the device casing module 1 can be configured to be disposed (or mounted) on the peripheral portion W2 of the steering wheel W of the transportation vehicle T (as shown in FIG. 2 and FIG. 3), or the device casing module 1 can be configured to be disposed (or mounted) on the central portion W1 of the steering wheel W of the transportation vehicle T (as shown in FIG. 4 and FIG. 5) according to different requirements. In addition, the device casing module 1 includes a casing structure 10, and the casing structure 10 can be provided on the central portion W1 or the peripheral portion W2 of the steering wheel W in an externally connected manner (as shown in FIG. 2 and FIG. 3, the casing structure 10 can be equipped with a clamp, buckle or fastener) or an embedded manner (as shown in FIG. 4 and FIG. 5). However, the aforementioned details are disclosed for exemplary purposes only, and are not meant to limit the scope of the present disclosure.

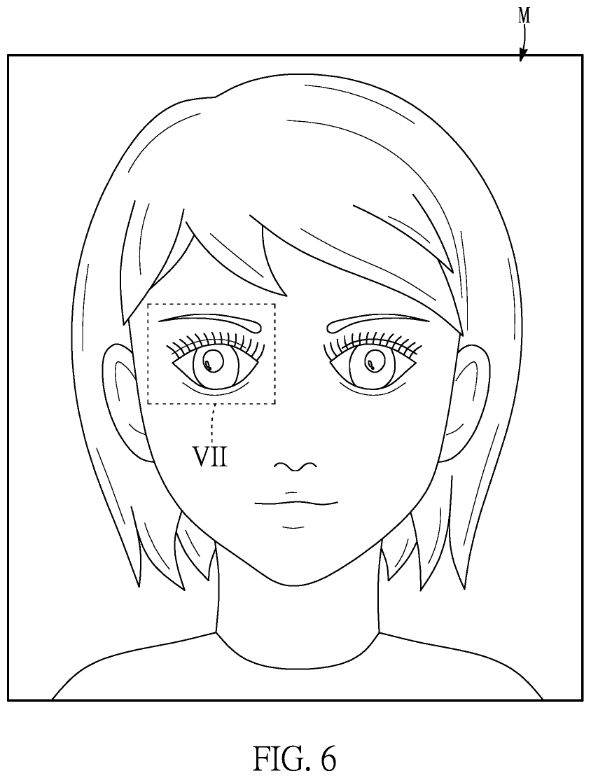

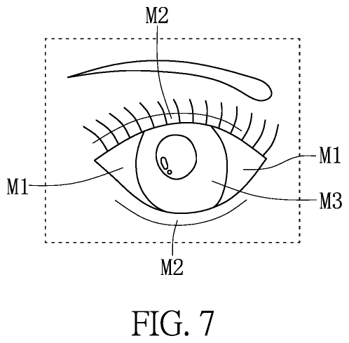

More particularly, referring to FIG. 1, FIG. 2, FIG. 6 and FIG. 7, the signal control module 2 can be disposed inside the device casing module 1, and the image capturing module 3 can be disposed inside the device casing module 1 (or a part of the image capturing module 3 can be exposed outside the device casing module 1) and electrically connected to the signal control module 2. For example, when the image capturing module 3 is optionally configured to be used, the image capturing module 3 can be allowed to be configured through the signal control module 2 to continuously (or continuously and regularly) or discontinuously (or non-continuously and regularly) capture a plurality of facial images M (or facial characteristic images) of a driver U driving the transportation vehicle T within a predetermined period (such as within tens of seconds or minutes), thereby obtaining a plurality of facial image signals S1 (or signals of facial characteristic images) respectively corresponding to the facial images M of the driver U, and each of the facial images M of the driver U includes at least one or more scleral images M1 (or images of white of the eye) with microvascular characteristics (such as capillaries in the scleral) or at least one or more eyelid images M2 (or eyelid area images each including an upper eyelid image and a lower eyelid image) with microvascular characteristics (such as capillaries in the skin). In addition, the number of the facial images M obtained by the image capturing module 3 within the predetermined period can exceed a predetermined value (for example, the number of the facial images M can be any positive integer between 10 and 200, or any positive integer exceeding 200), thereby obtaining blood flow changes (such as changes in blood flow velocity or blood flow conditions of the microvessels or capillaries between the scleral images M1 or between the eyelid images M2) or spectral changes (such as the spectral changes or dark line spectral changes in the microvessels or capillaries between the scleral images M1 or between the eyelid images M2, for example, spectral changes may include absorption spectrum changes, emission spectrum changes, scattering spectrum changes or any kind of spectral changes) in capillaries of scleras or eyelids of the driver U. More particularly, the image capturing module 3 includes at least one lower pixel facial image capturer 31 (such as a low-level pixel image sensor that can be configured to roughly find the eye position of the driver U so as to obtain a rough image such as an eye contour image or a facial contour image) and at least one higher pixel eye image capturer 32 (such as a high-level pixel image sensor that can be configured to clearly and correctly obtain an eye image of one or both eyes of the driver U), and the at least one lower pixel facial image capturer 31 can be configured to confirm an eye position range (such as including a facial contour and an eye contour) of the driver U to obtain eye position information, and the at least one higher pixel eye image capturer 32 can be configured to quickly and accurately capture the at least one scleral image M1 or the at least one eyelid image M2 of the driver U based on the eye position information provided by the at least one lower pixel facial image capturer 31. In addition, the image capturing module 3 includes an angle-adjustable lens structure 30 (such as an angle adjustment lens structure that can be adjusted arbitrarily within a predetermined angle range), and the angle-adjustable lens structure 30 can be configured to manually or automatically adjust an image capturing angle θ relative to the driver U. Therefore, when the image capturing module 3 is optionally configured to be used, the image capturing module 3 can be allowed to be configured through the signal control module 2 to manually or automatically (or continuously or discontinuously) adjust the image capturing angle θ of the angle-adjustable lens structure 30 relative to the driver U, until the facial image M of the driver U driving the transportation vehicle T is completely captured by the image capturing module 3 (as shown in FIG. 6). It should be noted that the signal control module 2 can use a central processing unit (CPU), a digital signal processor (DSP), a microprocessor (MPU), a microcontroller (MCU) or any type of control chip with any type of memory. In addition, the image capturing module 3 may include one or more image sensors or image readers, the image capturing module 3 may also include multiple image sensors or image readers of the same type or different types, and the image sensor used in the image capturing module 3 can be a charge-coupled device (CCD) image sensor, a complementary metal oxide semiconductor (CMOS) image sensor, or any type of image sensor. However, the aforementioned details are disclosed for exemplary purposes only, and are not meant to limit the scope of the present disclosure.

More particularly, referring to FIG. 1, FIG. 2, FIG. 3 and FIG. 6, the wireless transmission module 4 can be disposed inside the device casing module 1 and electrically connected to the signal control module 2. For example, when the wireless transmission module 4 is optionally configured to be used, the wireless transmission module 4 can be allowed to be configured through the signal control module 2 to wirelessly transmit the facial image signals S1 to an information processing system P (that is to say, the physiological state detection device D and the information processing system P can communicate with each other in a wireless manner), thereby obtaining a physiological state signal S2 (or physiological and mental state signals, or physical and mental state signals, which may include, for example, heart rate, blood pressure, blood oxygen, lactate, blood sugar, sleepiness and alcohol concentration, or any kind of physiological and mental information) corresponding to the facial image signals S1. In one possible embodiment, physiological information may include, for example, physical and mental reference indexes (such as physiological stress index, depression index, fatigue level, etc.), physiological monitoring values (such as heart rate, blood pressure, blood oxygen, lactate, blood sugar, etc.), and disease risk assessment factors (such as high blood pressure, heart disease, myocardial infarction, diabetes, Alzheimer's disease, Parkinson's disease, cancer, stroke probability, etc.). In addition, when the wireless transmission module 4 is optionally configured to be used, the wireless transmission module 4 can be allowed to be configured through the signal control module 2 to wirelessly receive the physiological state signal S2 that is obtained through processing by the information processing system P. More particularly, the wireless transmission module 4 can perform wireless data transmission through the cooperation of the antenna structure (or antenna chip) with Wi-Fi, Bluetooth, ZigBee or any wireless transmission method. Moreover, the information processing system P can be configured anywhere (such as a data processing center) away from the transportation vehicle T or can be installed directly inside the transportation vehicle T, the information processing system P at least includes a database and an information processing device, and the information processing device can be configured to compare and calculate the facial image signals S1 based on the database (or the big data), thereby obtaining the physiological state signal S2 (such as heart rate, blood pressure, blood oxygen, lactate, blood sugar, sleepiness and alcohol concentration). However, the aforementioned details are disclosed for exemplary purposes only, and are not meant to limit the scope of the present disclosure.

More particularly, referring to FIG. 1, FIG. 2 and FIG. 3, the information providing module 5 can be disposed inside the device casing module 1 (or a part of the information providing module 5 can be exposed outside the device casing module 1) and electrically connected to the signal control module 2. For example, when the information providing module 5 is optionally configured to be used, the information providing module 5 can be allowed to be configured through the signal control module 2 to present the physiological state signal S2 for reference by relevant personnel (such as the driver U driving the transportation vehicle T, the passengers riding in the transportation vehicle T, the remote controller of the transportation vehicle T, or the family members or the doctors away from the transportation vehicle T). In addition, the information providing module 5 can be allowed to be configured as an information display 51 (such as a transparent display) for visually presenting the physiological state signal S, an information projector 52 for visually presenting the physiological state signal S, a sound player 53 for audibly presenting the physiological state signal S2 and a vibration generator 54 for tangibly presenting the physiological state signal S2, or at least one or more of the information display 51, the information projector 52, the sound player 53 and the vibration generator 54 can cooperate with each other to present the physiological state signal S2. However, the aforementioned details are disclosed for exemplary purposes only, and are not meant to limit the scope of the present disclosure.

For example, as shown in FIG. 1, when the information providing module 5 is optionally configured to be used, the information providing module 5 can be allowed to be configured as at least one or more information displays 51 (such as data displays that can display different data, or light displays that can display lights of different colors) for displaying the physiological state signal S2, so that the information display 51 can be allowed to be configured to present (or display) the physiological state signal S2 (such as image signals) in a visible or visual manner (such as displaying numbers, text, images or colors) through the control of the signal control module 2 for reference by relevant personnel. Furthermore, when the information providing module 5 is optionally configured to be used, the information providing module 5 can be allowed to be configured as at least one or more information projectors 52 (such as small projectors) for projecting the physiological state signal S2 to a predetermined area (such as a windshield or a car dashboard), so that the information projector 52 can be allowed to be configured to present (or display) the physiological state signal S2 (such as image signals) in a visible or visual manner (such as displaying numbers, text, images or colors) through the control of the signal control module 2 for reference by relevant personnel. Moreover, when the information providing module 5 is optionally configured to be used, the information providing module 5 can be allowed to be configured as at least one or more sound players 53 (such as small speakers) for playing the physiological state signal S2, so that the sound player 53 can be allowed to be configured to present (or play) the physiological state signal S2 (such as audio signals) in an audible or auditory manner (such as playing pure tones or polyphonic tones, or musical tones or non-musical tones) through the control of the signal control module 2 for reference by relevant personnel. In addition, when the information providing module 5 is optionally configured to be used, the information providing module 5 can be allowed to be configured as at least one or more vibration generators 54 (such as small vibration motors) for generating different vibration frequencies based on or according to changes in the physiological state signal S2, so that the vibration generator 54 can be configured to present (or generate) the physiological state signal S2 (such as vibration signals) in a touchable or tactile manner (such as generating continuous or discontinuous vibrations, or generating high-frequency or low-frequency vibrations) through the control of the signal control module 2 for reference by relevant personnel. However, the aforementioned details are disclosed for exemplary purposes only, and are not meant to limit the scope of the present disclosure.

For example, referring to FIG. 1 and FIG. 2, when the wireless transmission module 4 is optionally configured to be used, the wireless transmission module 4 can be allowed to be configured through the signal control module 2 to wirelessly transmit the received physiological state signal S2 to a near-end information providing module N5 (such as a near-end information providing module provided by the transportation vehicle T, or another near-end information providing module provided by the portable device of the driver U) adjacent to the driver U. More particularly, when the near-end information providing module N5 is optionally configured to be used, the near-end information providing module N5 can be allowed to be configured as at least one or more near-end information displays N51 (such as a vehicle display N511 or a vehicle instrument panel N512 provided by the transportation vehicle T, or an image display N513 provided by the driver's portable device, e.g., a mobile phone, tablet or AR glasses) for displaying the physiological state signal S2, so that the near-end information display N51 can be allowed to be configured to present (or display) the physiological state signal S2 in a visible or visual manner for reference by relevant personnel. Furthermore, when the near-end information providing module N5 is optionally configured to be used, the near-end information providing module N5 can be allowed to be configured as a near-end information projector N52 (such as a head-up head (HUD) provided by the transportation vehicle T) for projecting the physiological state signal S2 to a predetermined area (such as a windshield), so that the near-end information projector N52 can be configured to present the physiological status signal S2 in a visible or visual way for the reference of relevant personnel. Moreover, when the near-end information providing module N5 is optionally configured to be used, the near-end information providing module N5 can be allowed to be configured as at least one or more near-end sound players N53 (such as a car audio N531 provided by the transportation vehicle T, or a speaker N532 provided by the driver's portable device, e.g., a mobile phone, tablet, AR glasses or wireless headphone) for playing the physiological state signal S2, so that the near-end sound player N53 can be allowed to be configured to present (or play) the physiological state signal S2 in an audible or auditory manner for reference by relevant personnel. In addition, when the near-end information providing module N5 is optionally configured to be used, the near-end information providing module N5 can be allowed to be configured as at least one or more near-end vibration generators N54 (such as a vibration motor provided by the driver's portable device, e.g., a mobile phone, tablet, AR glasses or wireless headphone) for generating different vibration frequencies based on or according to changes in the physiological state signal S2, so that the near-end vibration generator N54 can be allowed to be configured to present (or generate) the physiological state signal S2 in a touchable or tactile manner for reference by relevant personnel. It should be noted that, when the near-end information providing module N5 is optionally configured to be used, the near-end information providing module N5 can be allowed to be configured as an aroma generator or an odor generator (not shown in the figure, for example, the aroma generator or the odor generator can be mounted at the air-conditioning outlet of the transportation vehicle T or anywhere in the transportation vehicle T) for generating different aromas or odors based on changes in the physiological state signal S2, so that the aroma generator or the odor generator can be configured to present the physiological state signal S2 in a smellable or olfactory manner for reference by relevant personnel. In addition, when the near-end information providing module N5 is optionally configured to be used, the near-end information providing module N5 can be allowed to be configured as a temperature regulator or a temperature controller (such as an air-conditioning temperature controller of vehicle T, not shown in the figure) for generating different temperatures based on changes in the physiological state signal S2, so that the temperature regulator can be allowed to be configured to present the physiological state signal S2 through body sensing (such as the driver's temperature sensation or the driver's body sensation) for reference by relevant personnel. However, the aforementioned details are disclosed for exemplary purposes only, and are not meant to limit the scope of the present disclosure.

For example, as shown in FIG. 1, when the information processing system P can be configured to wirelessly transmit the processed physiological state signal S2 to a remote information providing module R5 (for example, it can be a portable electronic device such as a mobile phone, a tablet or a laptop, or a non-portable electronic device such as a desktop computer or a large system computer) away from the driver U, the remote information providing module R5 can be allowed to be configured as at least one or more remote information displays R51 (such as any kind of display) for displaying the physiological state signal S2, so that the remote information display R51 can be allowed to be configured to present (or display) the physiological state signal S2 in a visible or visual manner for reference by relevant personnel. Alternatively, when the information processing system P can be configured to wirelessly transmit the physiological state signal S2 to a remote information providing module R5 away from the driver U, the remote information providing module R5 can be allowed to be configured as at least one or more remote sound players R53 (such as any kind of speaker) for playing the physiological state signal S2, so that the remote sound player R53 can be allowed to be configured to present (or play) the physiological state signal S2 in an audible or auditory manner for reference by relevant personnel. Alternatively, when the information processing system P can be configured to wirelessly transmit the physiological state signal S2 to a remote information providing module R5 away from the driver U, the remote information providing module R5 can be allowed to be configured as at least one or more remote vibration generators R54 (such as any kind of vibration motor) for generating different vibration frequencies based on or according to changes in the physiological state signal S2, so that the remote vibration generator R54 can be allowed to be configured to present (or generate) the physiological state signal S2 in a touchable or tactile manner for reference by relevant personnel. However, the aforementioned details are disclosed for exemplary purposes only, and are not meant to limit the scope of the present disclosure.

More particularly, as shown in FIG. 1, the power supply module 6 can be disposed inside the device casing module 1 and electrically connected to the signal control module 2. For example, when the power supply module 6 is optionally configured to be used, the power supply module 6 can be allowed to be configured through the signal control module 2 to supply power to the signal control module 2, the image capturing module 3, the wireless transmission module 4 and the information providing module 5. In addition, the power supply module 6 can be any kind of rechargeable battery or solar cell. However, the aforementioned details are disclosed for exemplary purposes only, and are not meant to limit the scope of the present disclosure.

It should be noted that, for example, as shown in FIG. 1, the physiological state detection device D further includes an electrical connector module 7 disposed inside the device casing module 1 (or a part of the electrical connector module 7 can be exposed outside the device casing module 1) and electrically connected to the signal control module 2. More particularly, when the electrical connector module 7 is optionally configured to be used (such as using USB or any kind of transmission interface), the electrical connector module 7 can be allowed to be configured through the signal control module 2 to communicate with an external system in a wired manner (such as transmitting data or current). However, the aforementioned details are disclosed for exemplary purposes only, and are not meant to limit the scope of the present disclosure.

It should be noted that, for example, as shown in FIG. 1, the physiological state detection device D further includes an automatic light supplement module 8 (or an automatic light fill module) disposed inside the device casing module 1 (or a part of the automatic light supplement module 8 can be exposed outside the device casing module 1) and electrically connected to the signal control module 2. More particularly, the automatic light supplement module 8 includes an ambient light detector 81 and a light-filling component 82, and the light-filling component 82 can be a fill light for generating invisible light or visible light (or can be an IR LED fill light or an RGB LED fill light that can use any kind of LED, or can be a fill light that can use any kind of light-emitting element). Therefore, when the ambient light detector 81 is optionally configured to be used, the ambient light detector 81 can be allowed to be configured through the signal control module 2 to detect an ambient light around the driver U, thereby obtaining ambient light information. In addition, when the light-filling component 82 is optionally configured to be used, the light-filling component 82 can be allowed to be configured through the signal control module 2 to determine whether to provide a predetermined invisible light (or a predetermined visible light) to the face of the driver U based on whether the ambient light information (or ambient light conditions) meets a predetermined requirement, thereby improving the effect of the image capturing module 3 for capturing the facial images M (such as improving the contrast, brightness and clarity of the image). However, the aforementioned details are disclosed for exemplary purposes only, and are not meant to limit the scope of the present disclosure.

It should be noted that, for example, referring to FIG. 1, FIG. 2 and FIG. 7, the physiological state detection device D further includes a biometric module 9 disposed inside the device casing module 1 (or a part of the biometric module 9 can be exposed outside the device casing module 1) and electrically connected to the signal control module 2. More particularly, the biometric module 9 can be configured as an iris recognition module (or an iris identification module), a sclera recognition module (or a sclera identification module, such as for recognizing or identifying sclera blood vessels), a palmprint recognition module (or a palmprint identification module), a fingerprint recognition module (or a fingerprint identification module), a facial recognition module (or a facial identification module) or any kind of biometric module, or the biometric module 9 can also integrate the iris recognition module, the sclera recognition module, the palmprint recognition module, the fingerprint recognition module, the facial recognition module and any kind of biometric module at the same time. Therefore, when the biometric module 9 can be configured as the iris recognition module or the sclera recognition module, the iris recognition module or the sclera recognition module can be allowed to be configured through the signal control module 2 to capture at least one or more iris images M3 or sclera blood vessel images of the driver U (i.e., the iris image M3 or the sclera blood vessel image provided from the facial image M of the driver U), thereby identifying or determining (or confirming the identity of the driver U to determine) whether the driver U is qualified to use the physiological state detection device D. Moreover, when the biometric module 9 can be configured as the fingerprint recognition module or the palmprint recognition module, the fingerprint recognition module or the palmprint recognition module can be allowed to be configured through the signal control module 2 to capture at least one or more fingerprint images M4 or palmprint images of the driver U (i.e., the fingerprint image M4 or the palmprint image provided from the finger of the driver U), thereby identifying or determining (or confirming the identity of the driver U to determine) whether the driver U is qualified to use the physiological state detection device D. In addition, wherein, when the biometric module 9 can be configured as the facial recognition module, the facial recognition module can be allowed to be configured through the signal control module 2 to capture at least one facial image M of the driver U, thereby identifying or determining (or confirming the identity of the driver U to determine) whether the driver U is qualified to use the physiological state detection device D. However, the aforementioned details are disclosed for exemplary purposes only, and are not meant to limit the scope of the present disclosure.

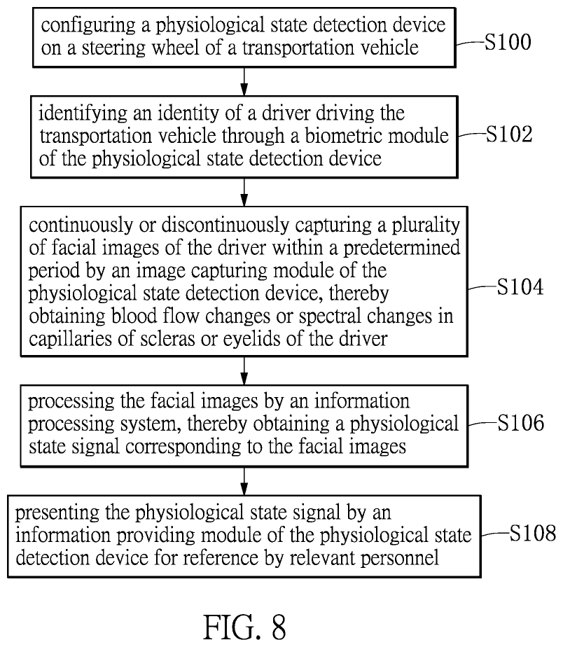

It should be noted that, referring to FIG. 1 to FIG. 8, the first embodiment of the present disclosure further provides a physiological state detection method, which includes: firstly, referring to FIG. 2, FIG. 4 and FIG. 8, configuring a physiological state detection device D on a steering wheel W of a transportation vehicle T such as in an externally connected manner or an embedded manner (step S100); next, referring to FIG. 1 and FIG. 8, identifying an identity of a driver U driving the transportation vehicle T through a biometric module 9 of the physiological state detection device D (step S102); then, referring to FIG. 1, FIG. 2, FIG. 6 and FIG. 8, continuously or discontinuously capturing a plurality of facial images M of the driver U within a predetermined period by an image capturing module 3 of the physiological state detection device D, thereby obtaining blood flow changes (such as changes in blood flow velocity or blood flow conditions of the microvessels or capillaries between the scleral images M1 or between the eyelid images M2) or spectral changes (such as absorption spectrum changes, emission spectrum changes, scattering spectrum changes or any kind of spectral changes) in capillaries of scleras or eyelids of the driver U, or thereby obtaining a plurality of facial image signals S1 respectively corresponding to the facial images M of the driver U (step S104); afterward, referring to FIG. 1, FIG. 6 and FIG. 8, processing the facial images M (or processing the facial image signals S1) by an information processing system P, thereby obtaining a physiological state signal S2 corresponding to the facial images M (step S106); and then referring to FIG. 1, FIG. 2 and FIG. 8, presenting (such as displaying, playing, generating) the physiological state signal S2 by an information providing module 5 of the physiological state detection device D for reference by relevant personnel (step S108). For example, when the information providing module 5 can be allowed to be configured as at least one or more information displays 51 for displaying the physiological state signal S2, the information display 51 can be allowed to be configured to visually present the physiological state signal S2. When the information providing module 5 can be allowed to be configured as at least one or more information projectors 52 for projecting the physiological state signal S2 to a predetermined area, the information projector 52 can be allowed to be configured to visually present the physiological state signal S2. When the information providing module 5 can be allowed to be configured as at least one or more sound players 53 for playing the physiological state signal S2, the sound player 53 can be allowed to be configured to audibly present the physiological state signal S2. When the information providing module 5 can be allowed to be configured as at least one or more vibration generators 54 for generating different vibration frequencies (or vibrations with different frequencies) based on changes in the physiological state signal S2, the vibration generator 54 can be configured to tangibly present the physiological state signal S2. However, the aforementioned details are disclosed for exemplary purposes only, and are not meant to limit the scope of the present disclosure.

### Second Embodiment

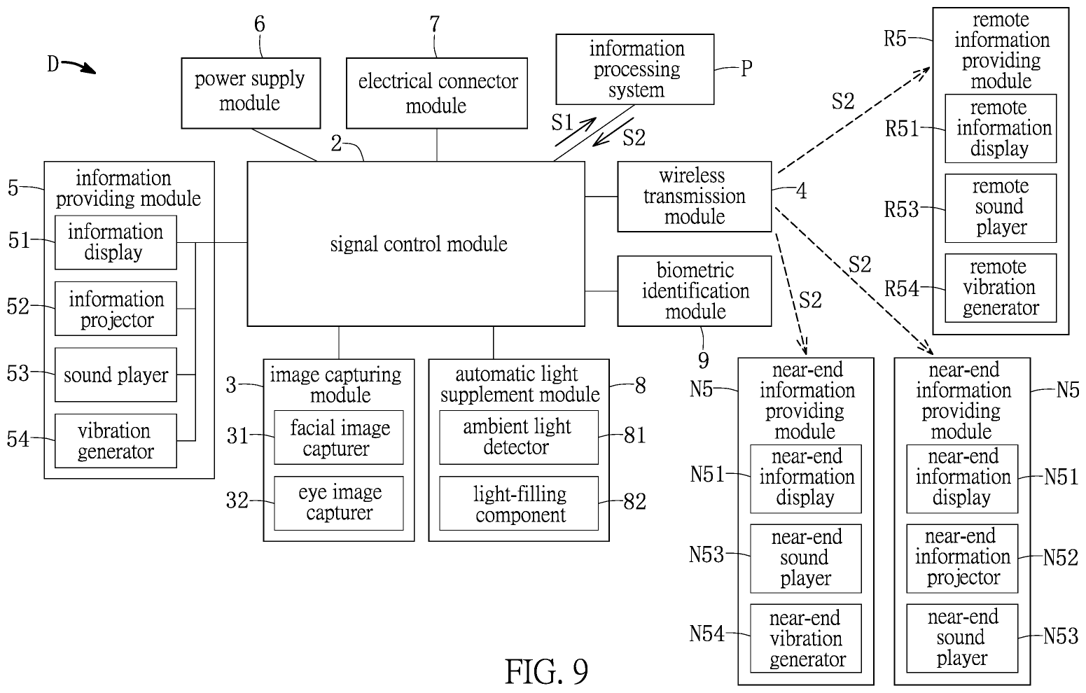

Referring to FIG. 2 to FIG. 9, a second embodiment of the present disclosure provides a physiological state detection device D (or a physiological and mental state detection device, or a physiological information detection device, or a physiological and mental information detection device) that can be applied to a transportation vehicle T, which includes a device casing module 1, a signal control module 2, an image capturing module 3, a wireless transmission module 4, an information providing module 5 and a power supply module 6. Comparing FIG. 9 with FIG. 1, the main difference between the second embodiment and the first embodiment is as follows: in the second embodiment, the information processing system P can be directly installed inside the transportation vehicle T, and the physiological state detection device D can be electrically connected to the information processing system P in a wired manner (that is to say, the physiological state detection device D and the information processing system P can communicate with each other in a wired manner). For example, when the image capturing module 3 can be configured to obtain a plurality of facial image signals S1 (or signals of facial characteristic images) respectively corresponding to a plurality of facial images M of the driver U through the control of the signal control module 2, the physiological state detection device D can be electrically connected to the information processing system P in a wired manner, so that the facial image signals S1 can be transmitted to an information processing system P in a wired manner for processing in order to obtain a physiological state signal S2 corresponding to the facial image signals S1, and the physiological state signal S2 that has been processed by the information processing system P can be transmitted back to the physiological state detection device D in a wired manner. It should be noted that the physiological state signal S2 that has been processed by the information processing system P can be wirelessly transmitted to a remote information providing module R5 (such as a portable electronic device, e.g., a mobile phone, a tablet or a laptop, or such as a non-portable electronic device, e.g., a desktop computer or a large system computer) away from the driver U through the wireless transmission module 4. However, the aforementioned details are disclosed for exemplary purposes only, and are not meant to limit the scope of the present disclosure.

### Beneficial Effects of the Embodiments

In conclusion, in the physiological state detection device D and the transportation vehicle T provided by the present disclosure, by virtue of “the image capturing module 3 being disposed inside the device casing module 1 and electrically connected to the signal control module 2” and “the information providing module 5 being disposed inside the device casing module 1 and electrically connected to the signal control module 2,” when the image capturing module 3 is optionally configured to be used, the image capturing module 3 can be allowed to be configured through the signal control module 2 to continuously or discontinuously capture a plurality of facial images M (each facial image includes at least one scleral image M1 with microvascular characteristics or at least one eyelid image M2 with microvascular characteristics) of a driver U driving the transportation vehicle T, and when the information providing module 5 is optionally configured to be used, the information providing module 5 can be allowed to be configured through the signal control module 2 to present a physiological state signal S2 corresponding to the facial images M for reference by relevant personnel.

Furthermore, in the physiological state detection method provided by the present disclosure, by virtue of “configuring a physiological state detection device D on a steering wheel W of a transportation vehicle T,” “identifying an identity of a driver U driving the transportation vehicle T through a biometric module 9 of the physiological state detection device D,” “continuously or discontinuously capturing a plurality of facial images M of the driver U within a predetermined period by an image capturing module 3 of the physiological state detection device D, thereby obtaining blood flow changes or spectral changes in/of capillaries of scleras or eyelids of the driver U” and “processing the facial images M by an information processing system P, thereby obtaining a physiological state signal S2 corresponding to the facial images M,” the physiological state signal S2 can be presented by an information providing module 5 of the physiological state detection device D for reference by relevant personnel. For example, when the information providing module 5 can be allowed to be configured as an information display 51 for displaying the physiological state signal S2, the information display 51 can be allowed to be configured to visually present the physiological state signal S2 for reference by relevant personnel. When the information providing module 5 can be allowed to be configured as an information projector 52 for projecting the physiological state signal S2 to a predetermined area, the information projector 52 can be allowed to be configured to visually present the physiological state signal S2 for reference by relevant personnel. When the information providing module 5 can be allowed to be configured as a sound player 53 for playing the physiological state signal S2, the sound player 53 can be allowed to be configured to audibly present the physiological state signal S2 for reference by relevant personnel. When the information providing module 5 can be allowed to be configured as a vibration generator 54 for generating different vibration frequencies based on changes in/of the physiological state signal S2, the vibration generator 54 can be configured to tangibly present the physiological state signal S2 for reference by relevant personnel.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A physiological state detection device applied to a transportation vehicle, comprising:
a device casing module configured to be disposed on a steering wheel of the transportation vehicle;
a signal control module disposed inside the device casing module;
an image capturing module disposed inside the device casing module and electrically connected to the signal control module;
a wireless transmission module disposed inside the device casing module and electrically connected to the signal control module;
an information providing module disposed inside the device casing module and electrically connected to the signal control module; and
a power supply module disposed inside the device casing module and electrically connected to the signal control module;
wherein, when the image capturing module is optionally configured to be used, the image capturing module is allowed to be configured through the signal control module to continuously or discontinuously capture a plurality of facial images of a driver driving the transportation vehicle within a predetermined period, thereby obtaining a plurality of facial image signals respectively corresponding to the facial images of the driver;
wherein, when the wireless transmission module is optionally configured to be used, the wireless transmission module is allowed to be configured through the signal control module to transmit the facial image signals to an information processing system, thereby obtaining a physiological state signal corresponding to the facial image signals;
wherein, when the wireless transmission module is optionally configured to be used, the wireless transmission module is allowed to be configured through the signal control module to receive the physiological state signal that is obtained through processing by the information processing system;
wherein, when the information providing module is optionally configured to be used, the information providing module is allowed to be configured through the signal control module to present the physiological state signal for reference by relevant personnel;
wherein, when the power supply module is optionally configured to be used, the power supply module is allowed to be configured through the signal control module to supply power to the signal control module, the image capturing module, the wireless transmission module and the information providing module;
wherein each of the facial images of the driver includes at least one scleral image with microvascular characteristics or at least one eyelid image with microvascular characteristics.

2. The physiological state detection device according to claim 1,
wherein the physiological state detection device further comprises an electrical connector module disposed inside the device casing module and electrically connected to the signal control module;
wherein, when the electrical connector module is optionally configured to be used, the electrical connector module is allowed to be configured through the signal control module to communicate with an external system in a wired manner;
wherein the physiological state detection device further comprises an automatic light supplement module disposed inside the device casing module and electrically connected to the signal control module, and the automatic light supplement module includes an ambient light detector and a light-filling component;
wherein, when the ambient light detector is optionally configured to be used, the ambient light detector is allowed to be configured through the signal control module to detect an ambient light around the driver, thereby obtaining ambient light information;
wherein, when the light-filling component is optionally configured to be used, the light-filling component is allowed to be configured through the signal control module to determine whether to provide a predetermined invisible light to the driver based on whether the ambient light information meets a predetermined requirement;
wherein the physiological state detection device further comprises a biometric module disposed inside the device casing module and electrically connected to the signal control module, and the biometric module is configured as an iris recognition module, a sclera recognition module, a palmprint recognition module, a fingerprint recognition module or a facial recognition module;
wherein, when the biometric module is configured as the iris recognition module or the sclera recognition module, the iris recognition module or the sclera recognition module is allowed to be configured through the signal control module to capture at least one iris image or sclera image of the driver, thereby identifying whether the driver is qualified to use the physiological state detection device;
wherein, when the biometric module is configured as the fingerprint recognition module or the palmprint recognition module, the fingerprint recognition module or the palmprint recognition module is allowed to be configured through the signal control module to capture at least one fingerprint image or palmprint image of the driver, thereby identifying whether the driver is qualified to use the physiological state detection device;
wherein, when the biometric module is configured as the facial recognition module, the facial recognition module is allowed to be configured through the signal control module to capture at least one facial image of the driver, thereby identifying whether the driver is qualified to use the physiological state detection device.

3. The physiological state detection device according to claim 1,
wherein the steering wheel of the transportation vehicle has a central portion and a peripheral portion surrounding the central portion;
wherein the device casing module includes a casing structure, and the casing structure is disposed on the central portion or the peripheral portion of the steering wheel in an external or embedded manner;
wherein the image capturing module includes at least one lower pixel facial image capturer and at least one higher pixel eye image capturer, and the at least one lower pixel facial image capturer is configured to confirm an eye position range of the driver to obtain eye position information, and the at least one higher pixel eye image capturer is configured to quickly and accurately capture the at least one scleral image or the at least one eyelid image of the driver based on the eye position information provided by the at least one lower pixel facial image capturer;
wherein the image capturing module includes an angle-adjustable lens structure, and the angle-adjustable lens structure is configured to manually or automatically adjust an image capturing angle relative to the driver;
wherein, when the image capturing module is optionally configured to be used, the image capturing module is allowed to be configured through the signal control module to manually or automatically adjust the image capturing angle of the angle-adjustable lens structure relative to the driver, until the facial image of the driver is completely captured by the image capturing module;
wherein the physiological state signal includes heart rate, blood pressure, blood oxygen, lactate, blood sugar, sleepiness and alcohol concentration;
wherein the number of the facial images obtained by the image capturing module within the predetermined period exceeds a predetermined value, thereby obtaining blood flow changes or spectral changes in capillaries of the driver's scleras or eyelids.

4. The physiological state detection device according to claim 1,
wherein, when the information providing module is optionally configured to be used, the information providing module is allowed to be configured as an information display for displaying the physiological state signal, so that the information display is allowed to be configured to visually present the physiological state signal;
wherein, when the wireless transmission module is optionally configured to be used, the wireless transmission module is allowed to be configured through the signal control module to transmit the physiological state signal to a near-end information providing module adjacent to the driver;
wherein, when the near-end information providing module is optionally configured to be used, the near-end information providing module is allowed to be configured as a near-end information display for displaying the physiological state signal, so that the near-end information display is allowed to be configured to visually present the physiological state signal;
wherein, when the information processing system is configured to transmit the physiological state signal to a remote information providing module away from the driver, the remote information providing module is allowed to be configured as a remote information display for displaying the physiological state signal, so that the remote information display is allowed to be configured to visually present the physiological state signal.

5. The physiological state detection device according to claim 1,
wherein, when the information providing module is optionally configured to be used, the information providing module is allowed to be configured as an information projector for projecting the physiological state signal to a predetermined area, so that the information projector is allowed to be configured to visually present the physiological state signal;
wherein, when the wireless transmission module is optionally configured to be used, the wireless transmission module is allowed to be configured through the signal control module to transmit the physiological state signal to a near-end information providing module adjacent to the driver;
wherein, when the near-end information providing module is optionally configured to be used, the near-end information providing module is allowed to be configured as a near-end information projector for projecting the physiological state signal to a predetermined area, so that the near-end information projector is allowed to be configured to visually present the physiological state signal;
wherein, when the near-end information providing module is optionally configured to be used, the near-end information providing module is allowed to be configured as an aroma generator for generating different aromas based on changes in the physiological state signal, so that the aroma generator is allowed to be configured to present the physiological state signal in a smellable manner;
wherein, when the near-end information providing module is optionally configured to be used, the near-end information providing module is allowed to be configured as a temperature regulator for generating different temperatures based on changes in the physiological state signal, so that the temperature regulator is allowed to be configured to present the physiological state signal through body sensing.

6. The physiological state detection device according to claim 1,
wherein, when the information providing module is optionally configured to be used, the information providing module is allowed to be configured as a sound player for playing the physiological state signal, so that the sound player is allowed to be configured to audibly present the physiological state signal;
wherein, when the wireless transmission module is optionally configured to be used, the wireless transmission module is allowed to be configured through the signal control module to transmit the physiological state signal to a near-end information providing module adjacent to the driver;
wherein, when the near-end information providing module is optionally configured to be used, the near-end information providing module is allowed to be configured as a near-end sound player for playing the physiological state signal, so that the near-end sound player is allowed to be configured to audibly present the physiological state signal;
wherein, when the information processing system is configured to transmit the physiological state signal to a remote information providing module away from the driver, the remote information providing module is allowed to be configured as a remote sound player for playing the physiological state signal, so that the remote sound player is allowed to be configured to audibly present the physiological state signal.

7. The physiological state detection device according to claim 1, wherein, when the information providing module is optionally configured to be used, the information providing module is allowed to be configured as a vibration generator for generating different vibration frequencies based on changes in the physiological state signal, so that the vibration generator is configured to tangibly present the physiological state signal;
wherein, when the wireless transmission module is optionally configured to be used, the wireless transmission module is allowed to be configured through the signal control module to transmit the physiological state signal to a near-end information providing module adjacent to the driver;
wherein, when the near-end information providing module is optionally configured to be used, the near-end information providing module is allowed to be configured as a near-end vibration generator for generating different vibration frequencies based on changes in the physiological state signal, so that the near-end vibration generator is allowed to be configured to tangibly present the physiological state signal;
wherein, when the information processing system is configured to transmit the physiological state signal to a remote information providing module away from the driver, the remote information providing module is allowed to be configured as a remote vibration generator for generating different vibration frequencies based on changes in the physiological state signal, so that the remote vibration generator is allowed to be configured to tangibly present the physiological state signal.

8. A physiological state detection device, comprising:
a device casing module;
a signal control module disposed inside the device casing module;
an image capturing module disposed inside the device casing module and electrically connected to the signal control module; and
an information providing module disposed inside the device casing module and electrically connected to the signal control module;
wherein, when the image capturing module is optionally configured to be used, the image capturing module is allowed to be configured through the signal control module to continuously or discontinuously capture a plurality of facial images of a driver within a predetermined period;
wherein, when the information providing module is optionally configured to be used, the information providing module is allowed to be configured through the signal control module to present a physiological state signal corresponding to the facial images;
wherein each of the facial images of the driver includes at least one scleral image with microvascular characteristics or at least one eyelid image with microvascular characteristics.

9. A transportation vehicle configured for using the physiological state detection device as claimed in claim 8,
wherein the physiological state detection device further comprises an electrical connector module disposed inside the device casing module and electrically connected to the signal control module;
wherein, when the electrical connector module is optionally configured to be used, the electrical connector module is allowed to be configured through the signal control module to communicate with an external system in a wired manner;
wherein the physiological state detection device further comprises an automatic light supplement module disposed inside the device casing module and electrically connected to the signal control module, and the automatic light supplement module includes an ambient light detector and a light-filling component;
wherein, when the ambient light detector is optionally configured to be used, the ambient light detector is allowed to be configured through the signal control module to detect an ambient light around the driver, thereby obtaining ambient light information;
wherein, when the light-filling component is optionally configured to be used, the light-filling component is allowed to be configured through the signal control module to determine whether to provide a predetermined invisible light to the driver based on whether the ambient light information meets a predetermined requirement;
wherein the physiological state detection device further comprises a biometric module disposed inside the device casing module and electrically connected to the signal control module, and the biometric module is configured as an iris recognition module, a sclera recognition module, a palmprint recognition module, a fingerprint recognition module or a facial recognition module;
wherein, when the biometric module is configured as the iris recognition module or the sclera recognition module, the iris recognition module or the sclera recognition module is allowed to be configured through the signal control module to capture at least one iris image or sclera image of the driver, thereby identifying whether the driver is qualified to use the physiological state detection device;
wherein, when the biometric module is configured as the fingerprint recognition module or the palmprint recognition module, the fingerprint recognition module or the palmprint recognition module is allowed to be configured through the signal control module to capture at least one fingerprint image or palmprint image of the driver, thereby identifying whether the driver is qualified to use the physiological state detection device;
wherein, when the biometric module is configured as the facial recognition module, the facial recognition module is allowed to be configured through the signal control module to capture at least one facial image of the driver, thereby identifying whether the driver is qualified to use the physiological state detection device;
wherein the image capturing module includes at least one lower pixel facial image capturer and at least one higher pixel eye image capturer, and the at least one lower pixel facial image capturer is configured to confirm an eye position range of the driver to obtain eye position information, and the at least one higher pixel eye image capturer is configured to quickly and accurately capture the at least one scleral image or the at least one eyelid image of the driver based on the eye position information provided by the at least one lower pixel facial image capturer;
wherein the image capturing module includes an angle-adjustable lens structure, and the angle-adjustable lens structure is configured to manually or automatically adjust an image capturing angle relative to the driver;
wherein, when the image capturing module is optionally configured to be used, the image capturing module is allowed to be configured through the signal control module to manually or automatically adjust the image capturing angle of the angle-adjustable lens structure relative to the driver, until the facial image of the driver is completely captured by the image capturing module;
wherein the physiological state signal includes heart rate, blood pressure, blood oxygen, lactate, blood sugar, sleepiness and alcohol concentration;
wherein the number of the facial images obtained by the image capturing module within the predetermined period exceeds a predetermined value, thereby obtaining blood flow changes or spectral changes in capillaries of the driver's scleras or eyelids;
wherein, when the information providing module is allowed to be configured as an information display for displaying the physiological state signal, the information display is allowed to be configured to visually present the physiological state signal;
wherein, when the information providing module is allowed to be configured as an information projector for projecting the physiological state signal to a predetermined area, the information projector is allowed to be configured to visually present the physiological state signal;
wherein, when the information providing module is allowed to be configured as a sound player for playing the physiological state signal, the sound player is allowed to be configured to audibly present the physiological state signal;
wherein, when the information providing module is allowed to be configured as a vibration generator for generating different vibration frequencies based on changes in the physiological state signal, the vibration generator is configured to tangibly present the physiological state signal;
wherein when the physiological state detection device is allowed to be configured to be electrically connected to an information processing system, the information processing system is allowed to be configured to process the facial images, thereby obtaining the physiological state signal corresponding to the facial images.

10. A physiological state detection method, comprising:
configuring a physiological state detection device on a steering wheel of a transportation vehicle;
identifying an identity of a driver driving the transportation vehicle through a biometric module of the physiological state detection device;
continuously or discontinuously capturing a plurality of facial images of the driver within a predetermined period by an image capturing module of the physiological state detection device, thereby obtaining blood flow changes or spectral changes in capillaries of the driver's scleras or eyelids;
processing the facial images by an information processing system, thereby obtaining a physiological state signal corresponding to the facial images; and
presenting the physiological state signal by an information providing module of the physiological state detection device for reference by relevant personnel;
wherein, when the information providing module is allowed to be configured as an information display for displaying the physiological state signal, the information display is allowed to be configured to visually present the physiological state signal;
wherein, when the information providing module is allowed to be configured as an information projector for projecting the physiological state signal to a predetermined area, the information projector is allowed to be configured to visually present the physiological state signal;
wherein, when the information providing module is allowed to be configured as a sound player for playing the physiological state signal, the sound player is allowed to be configured to audibly present the physiological state signal;
wherein, when the information providing module is allowed to be configured as a vibration generator for generating different vibration frequencies based on changes in the physiological state signal, the vibration generator is configured to tangibly present the physiological state signal.
